(19) United States
(12) Patent Application Publication
(10) Pub. No.: US 2016/0161443 A1
(43) Pub. Date: Jun. 9, 2016

(54) GAS SENSOR, METHOD FOR MANUFACTURING GAS SENSOR, AND METHOD FOR DETECTING GAS CONCENTRATION

(71) Applicant: Murata Manufacturing Co., Ltd., Nagaokakyo-shi, Kyoto-fu (JP)

(72) Inventor: Kazutaka Nakamura, Nagaokakyo-shi (JP)

(21) Appl. No.: 15/046,678

(22) Filed: Feb. 18, 2016

Related U.S. Application Data
(63) Continuation of application No. PCT/JP2014/065541, filed on Jun. 12, 2014.

(30) Foreign Application Priority Data
Aug. 30, 2013 (JP) 2013-179530

(51) Int. Cl. G01N 27/407; G01N 33/18

(52) U.S. Cl. CPC G01N 27/4071; G01N 33/18; G01N 2033/0095

(57) ABSTRACT

A humidity sensor that includes a p-type semiconductor layer and an n-type semiconductor layer on the p-type semiconductor layer. The p-type semiconductor layer is a sintered body made mainly of a solid solution of NiO and ZnO, and the n-type semiconductor layer is made mainly of at least one of ZnO and TiO₂. The p-type semiconductor layer has a molar ratio of Ni to Zn, or Ni/Zn, of 6/4 or more and 8/2 or less. The n-type semiconductor layer is produced using sputtering or through the firing of a multilayer structure composed of a green multilayer body to be made into the p-type semiconductor layer and a green sheet thereon to be made into the n-type semiconductor layer.

### CROSS REFERENCE TO RELATED APPLICATIONS

The present application is a continuation of International application No. PCT/JP2014/065541, filed Jun. 12, 2014, which claims priority to Japanese Patent Application No. 2013-179530, filed Aug. 30, 2013, the entire contents of each of which are incorporated herein by reference.

### FIELD OF THE INVENTION

The present invention relates to a gas sensor, a method for manufacturing a gas sensor, and a method for detecting a gas concentration. To be more specific, the present invention relates to a pn-junction gas sensor including p-type and n-type oxide semiconductor layers joined together at a heterojunction, a method for manufacturing this gas sensor, and a method for detecting the concentration of an ambient gas using this gas sensor.

### BACKGROUND OF THE INVENTION

There have been various forms proposed of humidity sensors, which detect the concentration of water vapor in the air, and other gas sensors.

For example, Non-patent Document 1 reports a gas sensor that uses an exposed junction (heterojunction) of semiconductors. The document describes the humidity-sensing characteristics of a pn-junction gas sensor composed of CuO, which is a p-type semiconductor, and ZnO, an n-type semiconductor.

At increased humidity levels, the pn-junction gas sensor described in Non-patent Document 1 experiences a great increase in the flow of current from the p-type semiconductor to the n-type semiconductor due to the rectifying effect in forward bias, while in reverse bias the current value remains substantially unchanged because charge is unlikely to be released in the reverse direction. The gas sensor uses this increase in current to detect humidity.

This type of pn-junction gas sensor exhibits a higher response rate than other gas sensors, and any water molecules physically adsorbing onto the contact interface in it are desorbed through electrolysis. A gas sensor of this type therefore requires no refreshing, cleaning of the contact interface by heating. Non-patent Document 1 also mentions another combination of p-type and n-type semiconductor layers, NiO and ZnO, in addition to CuO and ZnO.

Patent Document 1 proposes a junction chemical sensor. The junction chemical sensor has an upper electrode, a first member made of a first material and joined to the upper electrode, a second member made of a second material and joined to the first member, and a lower electrode joined to the second member. The interface at which the first and second members are joined together is exposed. This junction chemical sensor further has an AC voltage application unit for applying AC voltage across the upper and lower electrodes.

In Patent Document 1, for example, the p-type and n-type semiconductors are CuO and ZnO, respectively. P-type and n-type semiconductor layers are produced through a thin-film formation process, and the obtained p-type and n-type semiconductor layers are joined together.
Patent Document 1: Japanese Unexamined Patent Application Publication No. 5-264490
Non-patent Document 1: Handotai Seramikkusu (semiconductor ceramics), Section 4: Gas Sensors Using an Open Junction of Ceramic Semiconductors—Intellectualization of Ceramics by Masaru Miyayama, Kabushiki Kaisha TIC, Sep. 21, 1998, pp. 214-219

### SUMMARY OF THE INVENTION

In Non-patent Document 1 and Patent Document 1, however, the following problems are encountered because the p-type semiconductor material is CuO or NiO.

That is, when the p-type semiconductor material is a CuO-based material, prolonged operation can cause decomposition of part of CuO and diffusion of Cu ions over the surface of the n-type semiconductor layer. In such a situation, adhesion of Cu to the contact interface causes loss of characteristics and other problems, and the sensor is of low durability due to corrosion caused by the oxidation of the Cu itself.

When the p-type semiconductor material is a NiO-based material, it is customary to dope NiO with a monovalent alkali metal to make the NiO a semiconductor. This monovalent alkali metal, which acts as a strong alkali, promotes corrosion when diffused in NiO. In this case, too, the sensor is of low durability, and additionally is unsafe.

As mentioned in Patent Document 1, the p-type semiconductor layer of a pn-junction gas sensor of this type is usually produced using a thin-film formation process. Such a semiconductor layer is unstable at high temperatures compared to sintered bodies.

Made under these circumstances, the present invention is intended to provide a high-reliability and high-precision pn-junction gas sensor that offers good characteristics and high-temperature stability and excellent durability, a method for manufacturing a gas sensor, and a method for detecting a gas concentration.

After extensive research to achieve the above object, the inventor found the following. When the p-type and n-type semiconductor layers are a sintered body made mainly of (Ni, Zn)O containing predetermined proportions of Ni and Zn and a material made mainly of ZnO and/or TiO₂, respectively, the (Ni, Zn)O is stable in an oxidative atmosphere and serves as a semiconductor without requiring monovalent alkali metals. This gives the gas sensor good characteristics and high-temperature stability and excellent durability.

The present invention is based on these findings. A gas sensor according to the present invention includes a p-type semiconductor layer and an n-type semiconductor layer on the surface of the p-type semiconductor layer. The p-type semiconductor layer is a sintered body made mainly of a solid solution of NiO and ZnO, and the n-type semiconductor layer is made mainly of at least one of ZnO and TiO₂. This gas sensor is characterized in that the p-type semiconductor layer has a molar ratio of Ni to Zn, or Ni/Zn, of 6/4 or more and 8/2 or less. As a result, the p-type semiconductor layer is stable even in an oxidative atmosphere and serves as a semiconductor layer without requiring monovalent alkali metals. This gives the gas sensor good characteristics and high-temperature stability and excellent durability.

For the gas sensor according to the present invention, it is preferred that the p-type semiconductor layer contain at least one of Mn and a rare earth element. The quantity of the Mn relative to the NiO is less than 20 mol %, and that of the rare earth element relative to the NiO is less than 5 mol %.

This further reduces the specific resistance of the p-type semiconductor layer, giving the gas sensor higher sensitivity.

For the gas sensor according to the present invention, it is preferred that the Mn be in the form of peroxide.

For the gas sensor according to the present invention, it is preferred that the rare earth element include at least one selected from La, Pr, Nd, Sm, Dy, and Er.

For the gas sensor according to the present invention, furthermore, it is preferred that the n-type semiconductor layer is in such a configuration that part of the p-type semiconductor layer is exposed on a surface with an inner electrode embedded in the p-type semiconductor layer.

This makes it easy for gas molecules to physically adsorb onto the interface between the n-type and p-type semiconductor layers, allowing changes in resistance associated with electrolysis to be used to detect the concentration of the gas.

A method according to the present invention for manufacturing a gas sensor is characterized by including a shaped-article production step including producing a shaped article made mainly of a solid solution of NiO and ZnO, a firing step including firing the shaped article to obtain a p-type semiconductor layer as a sintered body, and a sputtering step including forming an n-type semiconductor layer on the surface of the p-type semiconductor layer by sputtering using a target material made mainly of at least one of ZnO and TiO₂. This method, utilizing sputtering to form an n-type semiconductor layer on a p-type semiconductor layer which is a sintered body, provides an easy way to obtain a gas sensor that offers good characteristics and high-temperature stability and excellent durability.

A method according to the present invention for manufacturing a gas sensor is characterized by including a shaped-article production step including producing a shaped article made mainly of a solid solution of NiO and ZnO, a sheet-shaped member production step including producing a sheet-shaped member made mainly of at least one of ZnO and TiO₂, a multilayer structure production step including placing the sheet-shaped member on a main surface of the shaped article to produce a multilayer structure, and a firing step including firing the multilayer structure to produce a sintered body wherein an n-type semiconductor layer is on a p-type semiconductor layer. In this method, therefore, the sheet-shaped member and the shaped article are sintered together. This method, too, provides an easy way to obtain a gas sensor that offers good characteristics and high-temperature stability and excellent durability.

A method according to the present invention for detecting a gas concentration includes detecting the concentration of an ambient gas using a gas sensor described in any of the foregoing. This method is characterized in that voltage is intermittently applied in pulses with the p-type and n-type semiconductor layers on the positive and negative electrode sides, respectively, and a current value measured at the application of the voltage is used to detect the gas concentration. This method therefore allows voltage to be applied according to the rate of adsorption of molecules of the gas onto the interface at which the p-type and n-type semiconductor layers are joined together, rendering the gas sensor highly reproducible.

### BRIEF DESCRIPTION OF THE DRAWINGS

FIG. 1 is a cross-sectional diagram schematically illustrating an embodiment of a humidity sensor as a gas sensor according to the present invention.

FIG. 2 is an exploded perspective view of a green multilayer body.

FIG. 3 is a diagram illustrating the method used in Examples to measure output current.

### DETAILED DESCRIPTION OF THE PREFERRED EMBODIMENTS

The following describes an embodiment of the present invention in detail with reference to the accompanying drawings.

FIG. 1 is a cross-sectional diagram illustrating an embodiment of a humidity sensor as a gas sensor according to the present invention.

This humidity sensor has a p-type semiconductor layer 1 and an n-type semiconductor layer 2, with the n-type semiconductor layer 2 joined to the p-type semiconductor layer 1 in such a configuration that part of the surface of the p-type semiconductor layer 1 is exposed. The p-type semiconductor layer 1 is a sintered body made mainly of a solid solution of NiO and ZnO, and the n-type semiconductor layer 2 is made of a ZnO-based material, a material the main component of which is ZnO.

There are first and second terminal electrodes 3a and 3b at both ends of the p-type semiconductor layer 1. To be more specific, there is an inner electrode 4 embedded in an upper section of the p-type semiconductor layer 1 with one of its ends exposed on the surface, and the first terminal electrode 3a is on one end portion of the p-type semiconductor layer 1 in such a manner as to be electrically coupled with the inner electrode 4. The second terminal electrode 3b is on the other end portion of the p-type semiconductor layer 1 in such a manner as to be electrically coupled with the n-type semiconductor layer 2.

The first and second terminal electrodes 3a and 3b are outer electrodes with their surface covered with first and second plating coatings. The outer electrodes are made of Ag or similar, the first plating coating is made of Ni or similar, and the second plating coating is made of Sn or similar.

The p-type semiconductor layer 1 can be represented by the general formula (Ni₁₋ₓZnₓ)O (hereinafter described as (Ni, Zn)O), and the molar proportion x of Zn is in the range of 0.2≦x≦0.4. If x is less than 0.2, the material can be highly resistive due to an excessively high Ni content. If x exceeds 0.4, ZnO particles can precipitate in crystal grain boundaries and make the material an n-type semiconductor due to an excessively high Zn content.

NiO and ZnO are therefore mixed in such a manner that the molar proportion x of Zn satisfies 0.2≦x≦0.4, i.e., the molar ratio of Ni to Zn, or Ni/Zn, is 6/4 or more and 8/2 or less.

The p-type semiconductor layer 1 only needs to be a (Ni, Zn)O-based sintered body, and it would be preferred that the layer contain trace amounts of additives. It is more preferred that the p-type semiconductor layer 1 contain an appropriate amount of Mn or a rare earth element in particular, because this contributes to lowering resistance by promoting additional increases in current. To be more specific, Mn or a rare earth element, when contained in the form of peroxide, acts to increase the valence of the divalent Ni oxide in the p-type semiconductor layer 1 by oxidizing it. The Ni oxide with an increased valance binds to oxygen, and this increases the number of carriers (holes and electrons). This provides a p-type semiconductor layer 1 with an even lower resistance value.

The Mn compound, a compound containing such Mn, can preferably be Mn₃O₄. The rare earth element can preferably be one selected from La, Pr, Nd, Sm, Dy, and Er or a combination of these.

If Mn is contained, its quantity needs to be less than 20 mol % relative to NiO. If the quantity of Mn is 20 mol % or more relative to NiO, an increased resistance value affects response sensitivity, and durability can also be impaired.

If a rare earth element is contained, too, its quantity needs to be less than 5 mol % relative to NiO because if the quantity of the rare earth element exceeds 5 mol % relative to NiO, an increased resistance value affects response sensitivity, and durability can also be impaired.

The ZnO-based material used to make the n-type semiconductor layer 2 may contain trace amounts of additives as long as its main component is ZnO. For example, dopants such as Al, Co, In, and Ga may be contained, and diffusing agents such as Fe, Ni, and Mn may be contained. Trace amounts of impurities such as Zr and Si do not compromise characteristics. In particular, adding dopants such as Al, Co, In, and Ga makes the resistance value even lower, thereby improving response sensitivity.

The material of which the inner electrode 4 is made, or the inner electrode material, is not limited. Examples of materials that can be used include a variety of metallic materials based on noble metals such as Pd, low-resistance composite oxides containing a rare earth element such as La with Ni, and so forth.

When voltage is applied across the first and second terminal electrodes 3a and 3b of a thus formed humidity sensor in forward bias with water molecules physically adsorbing onto the interface 7 at which the p-type and n-type semiconductor layers 1 and 2 are joined together (humidity-sensing section), the water molecules are electrolytically decomposed due to holes and electrons coming from the p-type and n-type semiconductor layers 1 and 2, respectively. This leads to a great increase in the flow of current from the p-type semiconductor layer 1 to the n-type semiconductor layer 2 as a result of the rectifying effect. Owing to the increase in current and the decrease in resistance after such electrolysis, the user can detect humidity by taking out a change in resistance as an electric signal. For example, when a bias voltage is intermittently applied in pulses in the forward direction at predetermined intervals (e.g., 1.5 seconds), the water molecules adhering to the contact interface 7 are electrolytically decomposed at each application of the voltage. While the voltage is not applied, the water molecules adhere to the contact interface 7 again. This allows the user to measure changes in resistance with good reproducibility, and thereby to detect the ambient humidity.

It is not preferred to apply a bias voltage continuously to this humidity sensor, even in the forward direction. To be more specific, when a bias voltage is applied in the forward direction continuously, the water molecules physically adsorbing onto the junction interface 7 are electrolyzed continuously. This causes the water molecules to detach from the contact interface 7, probably drying the junction interface 7 and increasing resistance. Continuous application of a bias voltage therefore affects response sensitivity and is not preferred. It is preferred that this humidity sensor perform detection in a place where the air flow rate is fast.

In this embodiment, the p-type semiconductor layer 1 is made mainly of (Ni, Zn)O. The (Ni, Zn)O is stable in oxidative atmospheres including the air and therefore causes less oxidation-related loss of characteristics than CuO-based materials. NiO-based materials require doping with a monovalent alkali metal, an element vulnerable to corrosion, to be a semiconductor, whereas the (Ni, Zn)O does not require doping with a monovalent alkali metal and therefore is free from corrosion caused by a monovalent alkali metal. As a result, the p-type semiconductor layer 1 has good corrosion resistance.

The p-type semiconductor layer 1 is, furthermore, a sintered body and therefore has better stability at high temperatures than if it were produced through a thin-film formation process.

Moreover, this humidity sensor has a faster response rate than other forms of humidity sensors, and water molecules diffuse off it through electrolysis. This allows the junction interface 7 to be kept in a constant dry state, making the humidity sensor highly usable.

This humidity sensor, which experiences an increase in current due to electrolysis in response to moisture, has been found not to respond ammonia or ethanol. This makes the humidity sensor a high-precision one with excellent gas selectivity.

The following describes a method for manufacturing this humidity sensor in detail.

[Production of a ZnO Sintered Body]

Predetermined amounts of a ZnO powder and optionally additives such as dopants and diffusing agents are weighed out. These apportioned materials are thoroughly wet-mixed and milled with a solvent such as purified water using a ball mill with cobblestones such as PSZ (partially stabilized zirconia) serving as milling medium. This yields a mixture in the form of slurry. This slurry mixture is dried by dehydration and then granulated into a predetermined particle diameter. The resulting particles are calcined at a predetermined temperature for approximately 2 hours, yielding a calcined powder. The obtained calcined powder is thoroughly wet-milled again with a solvent such as purified water using a ball mill with cobblestones serving as milling medium. This yields slurry of milled matter. This slurry of milled matter is dried by dehydration, and materials such as purified water, a dispersant, a binder, and a plasticizer are added to produce slurry for shaping. The slurry for shaping is then shaped into ZnO green sheets having a predetermined thickness using a shaping process such as doctor blading. A predetermined number of the ZnO green sheets are then stacked and pressure-bonded to produce a pressed article. This pressed article is degreased and then fired. In this way, a ZnO sintered body is obtained.

[Production of (Ni, Zn)O Green Sheets]

NiO and ZnO powders are weighed out to make the molar ratio of Ni to Zn, or Ni/Zn, in the range of 8/2 to 6/4. These apportioned materials are thoroughly wet-mixed and milled with a solvent such as purified water in a ball mill with cobblestones serving as milling medium. This yields a mixture in the form of slurry. This slurry mixture is dried by dehydration and then granulated into a predetermined particle diameter. The resulting particles are calcined at a predetermined temperature for approximately 2 hours, yielding a calcined powder. The obtained calcined powder is thoroughly wet-milled again with a solvent such as purified water in a ball mill with cobblestones serving as milling medium. This yields slurry of milled matter. This slurry of milled matter is dried by dehydration, and materials such as an organic solvent, a dispersant, a binder, and a plasticizer are added to produce slurry for shaping. The slurry for shaping is then shaped into (Ni, Zn)O green sheets having a predetermined thickness using a shaping process such as doctor blading.

[Production of Paste for the Formation of the Inner Electrode]

A binder resin is dissolved in an organic solvent in such a manner that, for example, the volume ratio of the binder resin to the organic solvent is in the range of 1:9 to 3:7, producing an organic vehicle. The binder resin is not limited and can be, for example, ethylcellulose resin, nitrocellulose resin, acrylic resin, alkyd resin, or a combination of these. The organic solvent is not limited either. Solvents such as α-terpineol, xylene, toluene, diethylene glycol monobutyl ether, diethylene glycol monobutyl ether acetate, diethylene glycol monoethyl ether, and diethylene glycol monoethyl ether acetate can be used alone or in combination.

Then, for example, a powder of a highly conductive metal, such as Pd, is mixed with the organic vehicle, and the resulting mixture is kneaded using a three-roll mill. In this way, paste for the formation of the inner electrode is produced.

[Production of a Green Multilayer Body]

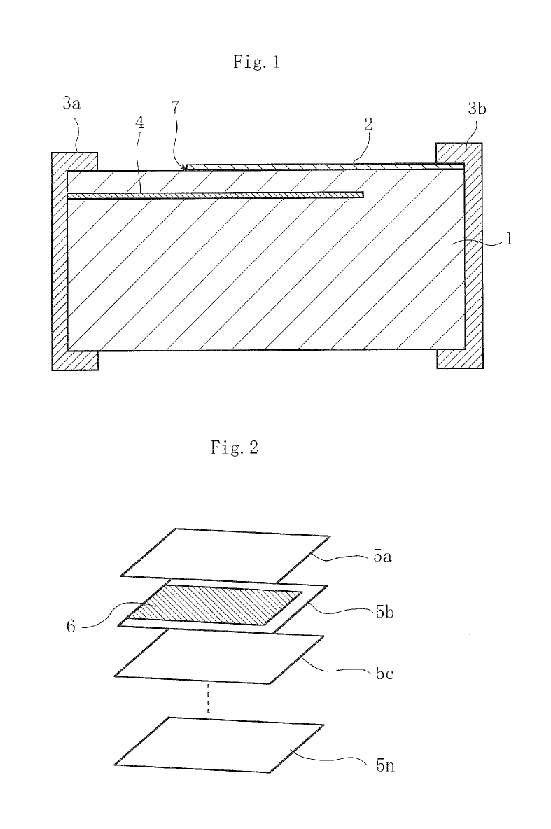

This section describes a method for producing a green multilayer body with reference to FIG. 2.

A predetermined number of (Ni, Zn)O green sheets 5a, 5b, 5c, . . . , and 5n are prepared, and the above-described paste for the formation of the inner electrode is applied to the surface of one (Ni, Zn)O green sheet 5b to form a conductive film 6.

Then a predetermined number of (Ni, Zn)O green sheets 5c to 5n having no conductive film are stacked. The (Ni, Zn)O green sheet 5b with the conductive film 6 is placed on this stack, and then a (Ni, Zn)O green sheet 5a having no conductive film is placed. The resulting stack is pressure-bonded to produce a green multilayer body.

[Production of the p-Type Semiconductor Layer 1]

The green multilayer body is thoroughly degreased and then fired at temperatures around 1200° C. for approximately 5 hours. This process of cofiring the conductive film 6 and the (Ni, Zn)O green sheets 5a to 5n yields a p-type semiconductor layer 1 in which an inner electrode 4 is embedded.

[Formation of the n-Type Semiconductor Layer 2]

With the ZnO sintered body serving as target, sputtering is performed using a metallic mask having a predetermined cavity to form an n-type semiconductor layer 2, a ZnO-based thin film, on the surface of the p-type semiconductor layer 1 in such a manner that part of the p-type semiconductor layer 1 is exposed on the surface.

[Production of Terminal Electrodes 3a and 3b]

Paste for the formation of outer electrodes is applied to both end portions of the p-type semiconductor layer 1 including the n-type semiconductor layer 2, and the applied paste is baked to form outer electrodes. The conductive material used in the paste for the formation of outer electrodes is not limited as long as it has good conductivity. Conductive materials such as Ag and Ag—Pd can be used.

Electrolytic plating is then performed to form a double-layer plating coating consisting of first and second plating coatings, forming first and second terminal electrodes 3a and 3b. In this way, a humidity sensor is obtained.

In this embodiment, therefore, a green multilayer body (shaped article) made mainly of (Ni, Zn)O is first produced. Then the green multilayer body is fired to produce a p-type semiconductor layer 1, and an n-type semiconductor layer 2 is formed on the surface of the p-type semiconductor layer 1 by sputtering using a ZnO sintered body as target material. This embodiment, utilizing sputtering to form the n-type semiconductor layer 2 on the p-type semiconductor layer 1 which is a sintered body, provides an easy way to obtain a humidity sensor that offers good humidity-sensing characteristics and high-temperature stability and excellent durability.

The present invention is not limited to the foregoing embodiment. Although in the above embodiment the n-type semiconductor layer 2 is a ZnO-based material, equivalent effects and advantages are obtained even if the ZnO-based material is replaced or used in combination with a TiO₂-based material, a material the main component of which is TiO₂.

In this case, the TiO₂-based material may contain trace amounts of additives as long as its main component is TiO₂. For example, it would be preferred that the material contain a dopant such as Nb. Adding such a dopant makes the resistance value even lower, thereby improving response sensitivity.

The TiO₂ sintered body for use as target material in sputtering can be produced using the same method and procedure as the ZnO sintered body described above.

Although in the above embodiment the n-type semiconductor layer 2 is produced using sputtering, it would be preferred to place a ZnO or TiO₂ green sheet cut into a predetermined size on a main surface of the aforementioned green multilayer body to produce a multilayer structure and then fire this multilayer structure to form the p-type semiconductor layer 1 and the n-type semiconductor layer through cofiring.

For the n-type semiconductor layer 2, it is preferred for improved response sensitivity at low humidity levels that a main surface of the p-type semiconductor layer 1 is sufficiently exposed with respect to the region where the n-type and p-type semiconductor layers 2 and 1 are joined together. For this purpose, it would be preferred that the n-type semiconductor layer 2 be in the shape of strips or similar.

The first and second terminal electrodes 3a and 3b may be a structure that covers the entire n-type semiconductor layer 2 as long as the aforementioned exposed portion is present. Such a structure reduces the resistance value of the series component, thereby improving response sensitivity.

Although the above embodiment exemplarily describes a humidity sensor, application to gas sensors that respond to gases other than water vapor in a similar way is also possible. Application to the detection of a variety of gases is made possible through the application of the detection method according to the present invention.

The following is a specific description of some examples of the present invention.

### Example 1

### Sample Number 1

[Production of a ZnO Sintered Body]

ZnO to serve as main component and Al₂O₃ as a dopant were weighed out to make the respective proportions in mol % 99.99 mol % and 0.01 mol %. These apportioned materials were mixed and milled together with purified water in a ball mill using PSZ beads as milling medium, yielding a mixture in the form of slurry with an average particle diameter of 0.5 μm or less. This slurry mixture was dried by dehydration and granulated to a particle diameter of approximately 50 μm. The resulting particles were calcined at a temperature of 1200° C. for 2 hours, yielding a calcined powder.

The thus obtained calcined powder was mixed and milled again together with purified water in a ball mill using PSZ beads as milling medium, yielding slurry of milled matter with an average particle diameter of 0.5 μm. This slurry of milled matter was dried by dehydration and then mixed together with an organic solvent and a dispersant. A binder and a plasticizer were then added to produce slurry for shaping, and green sheets having a thickness of 20 μm were produced using doctor blading. A predetermined number of these green sheets were then stacked to a thickness of 20 mm and pressure-bonded under a pressure of 250 MPa for 5 minutes to form a pressed article. This pressed article was degreased and then fired at a temperature of 1200° C. for 20 hours. In this way, a ZnO sintered body was obtained.

[Production of (Ni, Zn)O Green Sheets]

NiO and ZnO powders were weighed out to make the molar ratio Ni:Zn=7:3. These powders were then mixed and milled together with purified water using a ball mill with PSZ beads serving as milling medium, yielding a mixture in the form of slurry. This slurry mixture was dried by dehydration and granulated to a particle diameter of approximately 50 μm. The resulting particles were calcined at a temperature of 1200° C. for 2 hours, yielding a calcined powder. The thus obtained calcined powder was milled again together with purified water in a ball mill using PSZ beads as milling medium, yielding slurry of milled matter with an average particle diameter of 0.5 μm. This slurry of milled matter was dried by dehydration and mixed together with an organic solvent and a dispersant. A binder and a plasticizer were then added to produce slurry for shaping. This slurry for shaping was shaped into (Ni, Zn)O green sheets having a thickness of 10 μm using doctor blading.

[Paste for the Formation of the Inner Electrode]

Ethylcellulose resin as a binder resin and α-terpineol as an organic solvent were mixed in such a manner that the ethylcellulose resin and the α-terpineol constituted 30% by volume and 70% by volume, respectively, producing an organic vehicle. A Pd powder was then allowed to mix with the organic vehicle, and the resulting mixture was kneaded using a three-roll mill. In this way, paste for the formation of the inner electrode was produced.

[Production of a Green Multilayer Body]

The paste for the formation of the inner electrode was applied to the surface of one of the (Ni, Zn)O green sheets by screen printing. The applied paste was dried at a temperature of 60° C. for 1 hour to form a conductive film in a predetermined pattern.

Twenty (Ni, Zn)O green sheets having no conductive film were then stacked. The (Ni, Zn)O green sheet with the conductive film was placed on this stack, and then one (Ni, Zn)O green sheet having no conductive film was placed. These green sheets were pressure-bonded under a pressure of 20 MPa and then cut into a size of 2.1 mm×1.0 mm. In this way, a green multilayer body was produced.

[Production of a p-Type Semiconductor Layer]

The green multilayer body was thoroughly degreased at a temperature of 300° C. and then fired at a temperature of 1250° C. for 5 hours. In this way, a p-type semiconductor layer was obtained.

[Formation of an n-Type Semiconductor Layer]

With the ZnO sintered body serving as target material, sputtering was performed using a metallic mask to cover part of one main surface of the p-type semiconductor layer. In this way, an n-type semiconductor layer was produced in a predetermined pattern with a thickness of 0.5 μm.

[Production of Terminal Electrodes]

Ag paste was applied to both end portions of the p-type semiconductor layer including one end portion of the n-type semiconductor layer, and the applied paste was baked at a temperature of 800° C. to produce first and second outer electrodes. The surface of these first and second outer electrodes were electrolytically plated with a Ni coating and then a Sn coating to produce first and second terminal electrodes. In this way, a sample of sample number 1 was obtained.

### Sample Number 2

A ZnO green sheet having a thickness of 20 μm was produced using the same method and procedure as in [Production of a ZnO sintered body] for sample number 1 and cut into a predetermined size.

The ZnO green sheet was then placed on the green multilayer body produced for sample number 1. This structure was pressure-bonded at a pressure of 20 MPa and then cut into a size of 2.1 mm×1.0 mm. In this way, a multilayer structure was produced.

This multilayer structure was thoroughly degreased at a temperature of 300° C. and then fired at a temperature of 1250° C. for 5 hours so that the green multilayer body and the ZnO green sheet were sintered together. In this way, an n-type semiconductor layer was formed on a p-type semiconductor layer.

Then first and second terminal electrodes were formed using the same method and procedure as for sample number 1. In this way, a sample of sample number 2 was produced.

### Sample Number 3

A sample of sample number 3 was produced using the same method and procedure as for sample number 1, except that a TiO₂ sintered body was used to make the n-type semiconductor.

The TiO₂ sintered body was produced through the following process.

First, TiO₂ to serve as main component and Nb₂O₅ as a dopant were weighed out to make the respective proportions in mol % 99.0 mol % and 1.0 mol %. These apportioned materials were mixed and milled together with purified water in a ball mill using PSZ beads as milling medium, yielding a mixture in the form of slurry with an average particle diameter of 0.5 μm or less. This slurry mixture was dried by dehydration and granulated to a particle diameter of approximately 50 μm. The resulting particles were calcined at a temperature of 1200° C. for 2 hours, yielding a calcined powder.

The thus obtained calcined powder was mixed and milled again together with purified water in a ball mill using PSZ beads as milling medium, yielding slurry of milled matter with an average particle diameter of 0.5 μm. This slurry of milled matter was dried by dehydration and then mixed together with an organic solvent and a dispersant. A binder and a plasticizer were then added to produce slurry for shaping, and green sheets having a thickness of 20 μm were produced using doctor blading. A predetermined number of these green sheets were then stacked to a thickness of 20 mm and pressure-bonded under a pressure of 250 MPa for 5 minutes to give a pressed article. This pressed article was degreased and then fired at a temperature of 1200° C. for 20 hours, yielding a TiO₂ sintered body.

### Sample Number 4

A sample of sample number 4 was produced using the same method as sample number 2, except that a TiO₂ green sheet obtained in the course of producing the TiO₂ sintered body for sample number 3 was used, a green multilayer body was placed on the TiO₂ green sheet to produce a multilayer structure, and this multilayer structure was fired so that the green multilayer body and the TiO₂ green sheet were sintered together.

### Sample Numbers 5 to 8

Samples of sample numbers 5 to 8 were produced using the same method and procedure as for sample number 1, except that the (Ni, Zn)O green sheets contained MnO_{4/3} at 0.1 to 20 mol % relative to NiO.

### Sample Numbers 9 to 11

Samples of sample numbers 9 to 11 were produced using the same method and procedure as for sample number 1, except that the (Ni, Zn)O green sheets contained LaO_{3/2} at 0.1 to 5 mol % relative to NiO.

### Sample Numbers 12 to 16

Samples of sample numbers 12 to 16 were produced using the same method and procedure as for sample number 1, except that the (Ni, Zn)O green sheets contained PrO_{11/6}, NdO_{3/2}, SmO_{3/2}, DyO_{3/2}, or ErO_{3/2} each at 0.1 mol % relative to NiO.

### Sample Number 17

A sample of sample number 17 was produced using the same method and procedure as for sample number 1, except that the (Ni, Zn)O green sheets contained MnO_{4/3} and LaO_{3/2} each at 0.1 mol % relative to NiO.

### Sample Number 18

A sample of sample number 18 was produced using the same method and procedure as for sample number 1, except that NiO green sheets were used to make the p-type semiconductor layer.

The NiO green sheets were produced through the following process.

That is, NiO to serve as main component and Li₂O as a dopant were weighed out to make the respective proportions in mol % 99.0 mol % and 1.0 mol %. These apportioned materials were mixed and milled together with purified water using a ball mill with PSZ beads serving as milling medium, yielding a mixture in the form of slurry. This slurry mixture was dried by dehydration and granulated to a particle diameter of approximately 50 μm. The resulting particles were calcined at a temperature of 1200° C. for 2 hours, yielding a calcined powder. The thus obtained calcined powder was milled again together with purified water in a ball mill using PSZ beads as milling medium, yielding slurry of milled matter with an average particle diameter of 0.5 μm. This slurry of milled matter was dried by dehydration and then mixed together with an organic solvent and a dispersant. A binder and a plasticizer were then added to produce slurry for shaping. This slurry for shaping was shaped into NiO green sheets having a thickness of 10 μm using doctor blading.

[Evaluation of Samples]

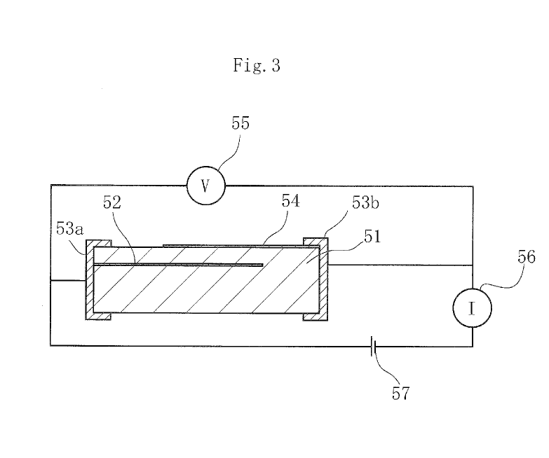

The samples of sample numbers 1 to 18 each have, as illustrated in FIG. 3, an inner electrode 52 embedded in a p-type semiconductor layer 51 with first and second terminal electrodes 53a and 53b at both ends of the p-type semiconductor layer 51 and an n-type semiconductor layer 54 on the surface of the p-type semiconductor layer 51 to be able to be electrically coupled with the second terminal electrode 53b. Each of these samples was placed in a thermo-hygrostat chamber, and a 1.5-V power supply 57 was placed between the first and second terminal electrodes 53a and 53b with the first terminal electrode 53a on the positive side and the second terminal electrode 53b on the negative side. A voltmeter 55 and an ammeter 56 were installed in the circuit.

The resistance value of the individual samples of sample numbers 1 to 18 was determined using the following method. That is, while a voltage of 1.5 V was applied across the first and second terminal electrodes 53a and 53b in the forward direction, and changes were made to control the thermo-hygrostat chamber to temperatures: 20° C. to 50° C. and relative humidity: 30% to 90%, the current values at the individual temperature and humidity conditions were measured with the ammeter 56. To be more specific, a voltage of 1.5 V was intermittently applied in pulses at 2-second intervals, the current value at 1.5 seconds after the application of voltage was measured with the ammeter 56, and the resistance was determined from this current value.

Furthermore, the durability of the individual samples of sample numbers 1 to 18 was assessed through the measurement of percent decrease in resistance using the following method.

The initial resistance of each sample was determined first. That is, the environment was controlled to a temperature of 30° C. and a relative humidity of 80%, a voltage of 1.5 V was intermittently applied in pulses at 2-second intervals, and the current value at 1.5 seconds after the application of voltage was measured with the ammeter 56. This measurement was used to determine the resistance at a temperature of 30° C. and a relative humidity of 80% as initial resistance.

The environment was then controlled to a temperature of 85° C. and a relative humidity of 95%. After the sample was left in this environmental atmosphere for 500 hours, the resistance value was determined from a current value using the same method and procedure as in the above derivation of initial resistance. The percent decrease in resistance was calculated from the initial resistance and the resistance after the sample was left, and the result was used to assess durability.

Table 1 summarizes main specifications of sample numbers 1 to 18 along with measurement results.

TABLE 1

| | | | | | | | | | | | Decrease |
|---|---|---|---|---|---|---|---|---|---|---|---|
| | | | | | | Resistance values (MΩ) | | | | | in |
| | P-type | | | N-type | | Temp., | Temp., | Temp., | Temp., | Temp., | resistance |
| | semiconductor layer | | | semiconductor layer | | 20° C.; | 20° C.; | 30° C.; | 50° C.; | 50° C.; | (at 500 |
| Sample | Main | | Content | Main | | hum., | hum., | hum., | hum., | hum., | hours) |
| No. | component | Additives | (mol %) | component | Process | 30% RH | 90% RH | 80% RH | 30% RH | 90% RH | (%) |
| 1 | (Ni, Zn)O | — | — | ZnO | Sputtering | 3.2 | 1.1 | 0.82 | 1.2 | 0.15 | 3.4 |
| 2 | (Ni, Zn)O | — | — | ZnO | Sintering | 4.2 | 1.2 | 0.94 | 2.2 | 0.24 | 2.8 |
| 3 | (Ni, Zn)O | — | — | TiO₂ | Sputtering | 4.2 | 1.6 | 0.89 | 0.8 | 0.072 | 1.4 |
| 4 | (Ni, Zn)O | — | — | TiO₂ | Sintering | 5.2 | 1.8 | 1.2 | 1.0 | 0.12 | 1.2 |
| 5 | (Ni, Zn)O | MnO_{4/3} | 0.1 | ZnO | Sputtering | 3.8 | 0.95 | 0.85 | 0.85 | 0.052 | 3.3 |
| 6 | (Ni, Zn)O | MnO_{4/3} | 1.0 | ZnO | Sputtering | 4.5 | 0.85 | 0.66 | 0.94 | 0.063 | 3.4 |
| 7 | (Ni, Zn)O | MnO_{4/3} | 10 | ZnO | Sputtering | 6.3 | 0.76 | 0.52 | 0.68 | 0.025 | 3.6 |
| 8** | (Ni, Zn)O | MnO_{4/3} | 20 | ZnO | Sputtering | 8.6 | 7.6 | 2.2 | 0.18 | 0.15 | 5.2 |
| 9 | (Ni, Zn)O | LaO_{3/2} | 0.1 | ZnO | Sputtering | 5.4 | 0.8 | 0.75 | 0.1 | 0.048 | 2.5 |
| 10 | (Ni, Zn)O | LaO_{3/2} | 1.0 | ZnO | Sputtering | 6.8 | 1.0 | 0.82 | 0.095 | 0.012 | 3.8 |
| 11** | (Ni, Zn)O | LaO_{3/2} | 5.0 | ZnO | Sputtering | 7.7 | 5.2 | 0.26 | 0.033 | 0.019 | 6.4 |
| 12 | (Ni, Zn)O | PrO_{11/6} | 0.1 | ZnO | Sputtering | 6.1 | 0.79 | 0.64 | 0.092 | 0.036 | 2.4 |
| 13 | (Ni, Zn)O | NdO_{3/2} | 0.1 | ZnO | Sputtering | 7.3 | 0.81 | 0.65 | 0.099 | 0.015 | 3.1 |
| 14 | (Ni, Zn)O | SmO_{3/2} | 0.1 | ZnO | Sputtering | 6.5 | 0.87 | 0.82 | 0.1 | 0.013 | 2.6 |
| 15 | (Ni, Zn)O | DyO_{3/2} | 0.1 | ZnO | Sputtering | 8.2 | 1.4 | 1.05 | 0.65 | 0.034 | 2.7 |
| 16 | (Ni, Zn)O | ErO_{3/2} | 0.1 | ZnO | Sputtering | 6.5 | 1.4 | 0.88 | 0.75 | 0.022 | 1.9 |
| 17 | (Ni, Zn)O | MnO_{4/3}/ | 0.1/ | ZnO | Sputtering | 6.1 | 1.1 | 0.84 | 0.11 | 0.027 | 2.2 |
| | | LaO_{3/2} | 0.1 | | | | | | | | |
| 18* | NiO | Li₂O | 1.0 | ZnO | Sputtering | 5.8 | 1.4 | 1.0 | 0.85 | 0.19 | 19.5 |

*Out of the scope of the present invention (Claim 1)
**Out of the scope of the present invention (Claim 2)

Sample number 18 had a p-type semiconductor layer made mainly of NiO, and this p-type semiconductor layer contained Li, an element vulnerable to corrosion. The percent decrease in resistance at 500 hours was 19.5%, demonstrating poor durability.

Sample numbers 1 to 17 had a p-type semiconductor layer made mainly of (Ni, Zn)O. These samples exhibited low resistance values of less than 10 MΩ under all measurement conditions, together with favorable percent decreases in resistance of less than 7%.

The p-type semiconductor layer of sample numbers 1 to 4 contained no additives, whereas that of sample numbers 5 to 17 contained Mn or/and a rare earth element as additives. These samples indicate that adding additives generally reduces resistance, providing a humidity sensor with enhanced sensitivity.

Sample number 8 contained an excessively large amount of MnO_{4/3}, 20 mol %, relative to NiO. The resistance value was increased at relatively low temperatures of 20° C. to 30° C., and the percent decrease in resistance exceeded 5%. This sample had generally low humidity-sensing characteristics and durability.

Sample number 11 contained an excessively large amount of LaO_{3/2}, 5 mol %, relative to NiO. The resistance value was increased at a low temperature of 20° C., and the percent decrease in resistance exceeded 5%. This sample had generally low humidity-sensing characteristics and low durability.

In conclusion, the following was found. Adding an appropriate amount of Mn or a rare earth element reduces the resistance value and limits the percent decrease in resistance to even lower levels of 5% or less. Making the molar quantity of Mn 20 mol % or more relative to NiO or that of the rare earth element 5 mol % or more relative to NiO, however, impairs humidity-sensing characteristics and durability. If Mn or a rare earth element is added to the (Ni, Zn)O, it is preferred that its molar quantity be less than 20 mol % relative to NiO for Mn and less than 5 mol % relative to NiO for rare earth elements.

### Example 2

### Production of Samples

### Sample Numbers 21 to 25

Samples of sample numbers 21 to 25 were produced using the same method and procedure as for sample number 1, except that the molar ratio of Ni to Zn, or Ni/Zn, in preparing the (Ni, Zn)O green sheets was matched to the proportions in Table 2.

### Sample Number 26

A sample of sample number 26 was produced using the same method and procedure as for sample number 1, except that the inner electrode material was LaNiO₃.

The production of LaNiO₃ was as follows.

That is, NiO and La₂O₃ powders were weighed out to a molar ratio of 2:1. These apportioned materials were mixed and milled together with purified water in a ball mill using PSZ beads as milling medium, yielding a mixture in the form of slurry. This slurry mixture was dried by dehydration and granulated to a particle diameter of approximately 50 μm. The resulting particles were calcined at a temperature of 1200° C. for 2 hours, yielding a calcined powder. The calcined powder thus obtained was milled again together with purified water in a ball mill using PSZ beads as milling medium, yielding slurry of milled matter with an average particle diameter of 0.5 μm, and this slurry of milled matter was dried by dehydration. In this way, a LaNiO₃ powder was obtained.

[Evaluation of Samples]

For each of the samples of sample numbers 21 to 26, the resistance and percent decrease in resistance were measured under different temperature and humidity conditions using the same method and procedure as in Example 1.

Table 2 summarizes the measurement results.

TABLE 2

| | | | | | | | | Decrease |
|---|---|---|---|---|---|---|---|---|
| | | | Resistance values (MΩ) | | | | | in |
| | | | Temp., | Temp., | Temp., | Temp., | Temp., | resistance |
| | Ni/Zn | | 20° C.; | 20° C.; | 30° C.; | 50° C.; | 50° C.; | (at 500 |
| Sample | (molar | Inner | hum., | hum., | hum., | hum., | hum., | hours) |
| No. | ratio) | electrode | 30% RH | 90% RH | 80% RH | 30% RH | 90% RH | (%) |
| 21* | 9/1 | Pd | 195 | 185 | 95 | 65 | 48 | — |
| 22 | 8/2 | Pd | 12.5 | 3.6 | 2.8 | 1.4 | 0.32 | 4.2 |
| 23 | 7/3 | Pd | 3.2 | 1.1 | 0.82 | 1.2 | 0.15 | 3.4 |
| 24 | 6/4 | Pd | 2.4 | 0.65 | 0.55 | 0.39 | 0.024 | 2.8 |
| 25* | 5/5 | Pd | 0.8 | 0.65 | 0.55 | 0.25 | 0.21 | — |
| 26 | 7/3 | LaNiO₃ | 5.2 | 0.99 | 1.75 | 0.95 | 0.085 | 2.2 |

*Out of the scope of the present invention (Claim 1)

Sample numbers 21 to 25 represent samples in which the inner electrode material was Pd and the molar ratio of Ni to Zn, or Ni/Zn, varied.

Sample number 21 had a molar ratio of Ni to Zn, or Ni/Zn, of 9/1. This molar quantity of Ni was excessively large and led to high resistance.

Sample number 25 had a molar ratio of Ni to Zn, or Ni/Zn, of 5/5. This made the (Ni, Zn)O layer an n-type semiconductor and prevented it from serving as a humidity sensor.

Sample numbers 22 to 24 had a molar ratio of Ni to Zn, or Ni/Zn, in the scope of the present invention, 8/2 to 6/4. These samples had desired low resistance even under high humidity conditions, together with favorable percentage decreases in resistance of 2.8% to 4.2%.

Sample number 26 had an inner electrode made of LaNiO₃. As is clear from comparison with sample number 23, the use of this material made the percentage decrease in resistance even smaller.

The present invention makes possible a high-reliability and high-precision pn-junction gas sensor that offers good characteristics and high-temperature stability and excellent durability, a method for manufacturing this gas sensor, and a method for detecting a gas concentration.

### REFERENCE SIGNS LIST

1 P-type semiconductor layer
2 N-type semiconductor layer
4 Inner electrode

1. A gas sensor comprising:
a p-type semiconductor layer and an n-type semiconductor layer on a surface of the p-type semiconductor layer, the p-type semiconductor layer being a sintered body made mainly of a solid solution of NiO and ZnO and the n-type semiconductor layer made mainly of at least one of ZnO and TiO₂, wherein
the p-type semiconductor layer has a molar ratio of Ni to Zn of 6/4 or more and 8/2 or less.

2. The gas sensor according to claim 1, wherein the p-type semiconductor layer contains at least one of Mn and a rare earth element.

3. The gas sensor according to claim 2, wherein a quantity of the Mn relative to the NiO is less than 20 mol %.

4. The gas sensor according to claim 2, wherein a quantity of the rare earth element relative to the NiO is less than 5 mol %.

5. The gas sensor according to claim 1, wherein the p-type semiconductor layer contains Mn and a rare earth element,
a quantity of the Mn relative to the NiO is less than 20 mol %, and
a quantity of the rare earth element relative to the NiO is less than 5 mol %.

6. The gas sensor according to claim 5, wherein the Mn is in a form of a peroxide.

7. The gas sensor according to claim 6, wherein the rare earth element includes at least one selected from La, Pr, Nd, Sm, Dy, and Er.

8. The gas sensor according to claim 5, wherein the rare earth element includes at least one selected from La, Pr, Nd, Sm, Dy, and Er.

9. The gas sensor according to claim 2, wherein the Mn is in a form of a peroxide.

10. The gas sensor according to claim 2, wherein the rare earth element includes at least one selected from La, Pr, Nd, Sm, Dy, and Er.

11. The gas sensor according to claim 1, further comprising a first and a second terminal electrode on respective ends of the p-type semiconductor layer.

12. The gas sensor according to claim 1, wherein the n-type semiconductor layer does not completely cover the surface of the p-type semiconductor layer such that part of the p-type semiconductor layer is exposed, and the gas sensor further comprises an electrode embedded in the p-type semiconductor layer.

13. The gas sensor according to claim 12, further comprising a first and a second terminal electrode on respective ends of the p-type semiconductor layer.

14. The gas sensor according to claim 13, wherein the first terminal electrode is electrically coupled to the electrode embedded in the p-type semiconductor layer, and the second terminal electrode is electrically coupled to the n-type semiconductor layer.

15. A method for manufacturing a gas sensor, the method comprising:
producing a shaped article made mainly of a solid solution of NiO and ZnO,
firing the shaped article to obtain a p-type semiconductor layer as a sintered body, and
forming an n-type semiconductor layer on a surface of the p-type semiconductor layer by sputtering using a target material made mainly of at least one of ZnO and TiO₂.

16. The method for manufacturing the gas sensor according to claim 15, wherein
the p-type semiconductor layer contains at least one of Mn and a rare earth element,
a quantity of the Mn relative to the NiO is less than 20 mol %, and
a quantity of the rare earth element relative to the NiO is less than 5 mol %.

17. A method for manufacturing a gas sensor, the method comprising” producing a shaped article made mainly of a solid solution of NiO and ZnO,
producing a sheet-shaped member made mainly of at least one of ZnO and TiO₂,
placing the sheet-shaped member on a main surface of the shaped article to produce a multilayer structure, and
firing the multilayer structure to produce a sintered body having an n-type semiconductor layer on a p-type semiconductor layer.

18. The method for manufacturing the gas sensor according to claim 17, wherein
the p-type semiconductor layer contains at least one of Mn and a rare earth element,
a quantity of the Mn relative to the NiO is less than 20 mol %, and
a quantity of the rare earth element relative to the NiO is less than 5 mol %.

19. A method for detecting a gas concentration, the method comprising:
detecting a concentration of an ambient gas using a gas sensor according to claim 1 by applying voltage intermittently in pulses with the p-type and n-type semiconductor layers on positive and negative electrode sides, respectively, and using a current value measured at application of the voltage to detect the concentration of the ambient gas.
